(19) United States
(12) Patent Application Publication
(10) Pub. No.: US 2012/0087966 A1
(43) Pub. Date: Apr. 12, 2012

(54) ANTIMICROBIAL FOAM

(75) Inventor: Jan Frances HAVILAND, Westphalia, MO (US)

(73) Assignee: OBE Goods, LLC, Linn, MO (US)

(21) Appl. No.: 13/271,012

(22) Filed: Oct. 11, 2011

Related U.S. Application Data
(60) Provisional application No. 61/391,775, filed on Oct. 11, 2010.

(51) Int. Cl. A01N 25/16; A61K 9/12; A61P 31/00; B08B 1/00; A61L 15/22; A01P 1/00; C08J 9/12; B82Y 40/00; B82Y 5/00; B82Y 99/00

(52) U.S. Cl. 424/409; 264/54; 15/244.4; 602/46; 602/48; 977/700; 977/904; 977/902; 977/888

(57) ABSTRACT

A resilient foam material having an active antimicrobial bound to, incorporated within, and projecting from its surface is provided for incapacitating or destroying microbes. The antimicrobial has an atomic structure that is capable of mechanically piercing or lysing a microbe thereby incapacitating or destroying the microbe. The resilient antimicrobial foam material may be manufactured into a resilient antimicrobial foam product for drawing across a surface and mechanically incapacitating or destroying microbes on the surface. The resilient antimicrobial foam material may be manufactured into a membrane for providing a sterile barrier for a surface. The resilient antimicrobial foam material is manufactured by combining the antimicrobial with a foam polymer material, heating the foam polymer material under pressure to a temperate that does not deactivate the antimicrobial, incorporating a blowing agent, cooling the material, and extruding the material.

### CROSS-REFERENCE TO RELATED APPLICATIONS

This application claims priority in U.S. Provisional Patent Application No. 61/391,775, filed Oct. 11, 2010, which is incorporated herein by reference.

### BACKGROUND

The present invention relates generally to antimicrobial materials, and more specifically to a foam material having an antimicrobial nanostructure.

Foam material, including sponges, cleaning pads, blades for squeegees, and the like, are used to clean various surface, often in conjunction with one or more cleaning agents applied directly to the foam or surface. The cleaning agents used act either to chemically or mechanically incapacitate or destroy microbial agents.

Cleaning agents applied to the surface to be cleaned are typically required to be left in contact with the surface for a length of time to have the desired effect upon the microbial agents present. Applying a cleaning agent to a foam material, and passing the foam material and agent across the surface transfers the cleaning agent from the foam material to the surface resulting in an uneven distribution of the cleaning agent. Moreover, this approach results in removal of the cleaning agent from the foam material requiring reapplication of the cleaning agent and excessive use of the cleaning agent in order to have the desired antimicrobial effect.

The composition or characteristics of some cleaning agents makes them incompatible for use on some surfaces because they may damage the surfaces. Additionally, some cleaning agents have components that have undesirable side effects that make them undesirable for use in a variety of settings including off-gassing, odors, toxicity, as an irritant, and as an allergen.

### SUMMARY

A resilient foam material having an active antimicrobial incorporated with, bound to, and projecting from its surface is provided for incapacitating or destroying microbes. The antimicrobial has an atomic structure that is capable of mechanically piercing or lysing a microbe thereby incapacitating or destroying the microbe. The resilient antimicrobial foam material may be manufactured into a resilient antimicrobial foam product for drawing across a surface and mechanically incapacitating or destroying microbes on the surface. The resilient antimicrobial foam material may be manufactured into a membrane for providing a sterile barrier for a surface.

The resilient antimicrobial foam material is manufactured by combining the antimicrobial with a foam polymer material, heating the foam polymer material under pressure to a temperate between about 300 to about 350 degrees Fahrenheit to form a melt, incorporating a blowing agent, cooling the material creating a cool melt or bun that will be manufactured into various foam products having various sizes and dimensions.

### BRIEF DESCRIPTION OF THE DRAWINGS

The drawings constitute a part of this specification and include exemplary embodiments of the disclosed subject matter and illustrate various objects and features thereof.

FIG. 1 shows a resilient antimicrobial foam material embodying principles of the disclosed subject matter.

FIG. 2 is an enlarged view of the resilient antimicrobial foam material embodying principles of the disclosed subject matter taken generally within circle 2 in FIG. 1.

### DETAILED DESCRIPTION

As required, detailed aspects of the disclosed subject matter are disclosed herein; however, it is to be understood that the disclosed aspects are merely exemplary of the disclosed subject matter, which may be embodied in various forms. Therefore, specific structural and functional details disclosed herein are not to be interpreted as limiting, but merely as a basis for the claims and as a representative basis for teaching one skilled in the art how to variously employ the disclosed technology in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Said terminology will include the words specifically mentioned, derivatives thereof, and words of similar meaning.

Resilient foam materials are substances formed from the vaporization of gas within a matrix wherein the gas forms bubbles or cells disposed throughout the substance. The bubbles are typically formed by blowing a gas through a molten mixture. Polymer foams formed from polymer materials, and similar resilient foam materials that have cells formed from bubbles include latex, polyurethane, foam rubber, polyester, polystyrene, silicone rubber, neoprene, nitrite foam, and neoprene/ethylene propylene diene terpolymer blends. Products composed of resilient foams include sponges, cleaning pads, blades for squeegees, and the like.

Polymer foams may be produced by extrusion. In an extrusion process, the starting polymer material is first melted and the resulting molten or hot mixture is pressurized in a primary extruder, followed by the addition and incorporation of a blowing agent or agents that will create the cells. Blowing agents include hydrocarbons, halohydrocarbons, and inert gasses. The resulting melted polymer mixture is then cooled in a secondary extruder forming a cool melt and is delivered through a die. As the polymer mixture exits the die, the blowing agent vaporizes trapping the incorporated blowing agents within the polymer mixture creating a resilient foam material with cells disposed therein.

Polymer foam rubbers, including neoprene foams may be produced by blowing a gas through a neoprene latex mixture both before and during vulcanization forming a resilient foam material having cells disposed throughout.

Various additives may be incorporated in the polymer foams at the outset to give the resulting resilient foam material desirable characteristics. Such additives may include colorants, stabilizers, nucleators, flame retardants, plasticizers, nanoparticles, nanostructure antimicrobials, and the like.

There are nanostructure antimicrobials that have an atomic physical structure that enables them to incapacitate or destroy microbes. A nanostructure antimicrobial includes BioShield® (IndusCo. Ltd.; Greensboro, N.C.). BioShield® includes a quaternary ammonium salt chemically spliced to a silane molecule, resulting in 3-(trimethoxsilyl) propyldimethyl octadecyl ammonium chloride. The aforementioned atomic nanostructure characteristic of the antimicrobial is the physical structure that is capable of piercing or lysing a microbe thereby incapacitating or destroying the microbe. Accordingly, use of an antimicrobial that affects microbes by physical means avoids the need to use antimicrobials that affect microbes using chemical means. Physically, rather than chemically, incapacitating or destroying microbes reduces the development of resistance to incapacitation or destruction from adaptation, evolution, or mutation. By example, such a resistant type of microbe includes a multi-antibiotic resistant bacteria or a superbug, but would not be resistant to physical means of control.

Upon mixing the above nanostructure antimicrobial with the foam polymer during the manufacturing process it was discovered that the nano structure of the antimicrobial was unexpectedly maintained in the resulting resilient foam material thereby creating a polymer foam material having antimicrobial properties. It has been found that if the heating temperature of the manufacturing process, or the curing temperature of the curing process remains between about 300 to about 350 degrees Fahrenheit, the antimicrobial properties of the nanostructure remain unaffected, and the BioShield® antimicrobial becomes dispersed throughout and bound to the resilient foam material. It has been found that the addition of a nanostructure antimicrobial to the foam polymer during the manufacturing process such that the resulting resilient foam material contains one percent antimicrobial by unit volume results in a resilient foam material that presents active antimicrobial properties, and that does not lose antimicrobial potency over time. The resulting resilient antimicrobial foam material has the ability to remove, incapacitate, or destroy microbes on a surface by physical means due to the active antimicrobial nanostructure remaining within the foam structure, as well as the nanostructure antimicrobial being attached to and projecting outwardly from any available surface of the resilient antimicrobial foam material.

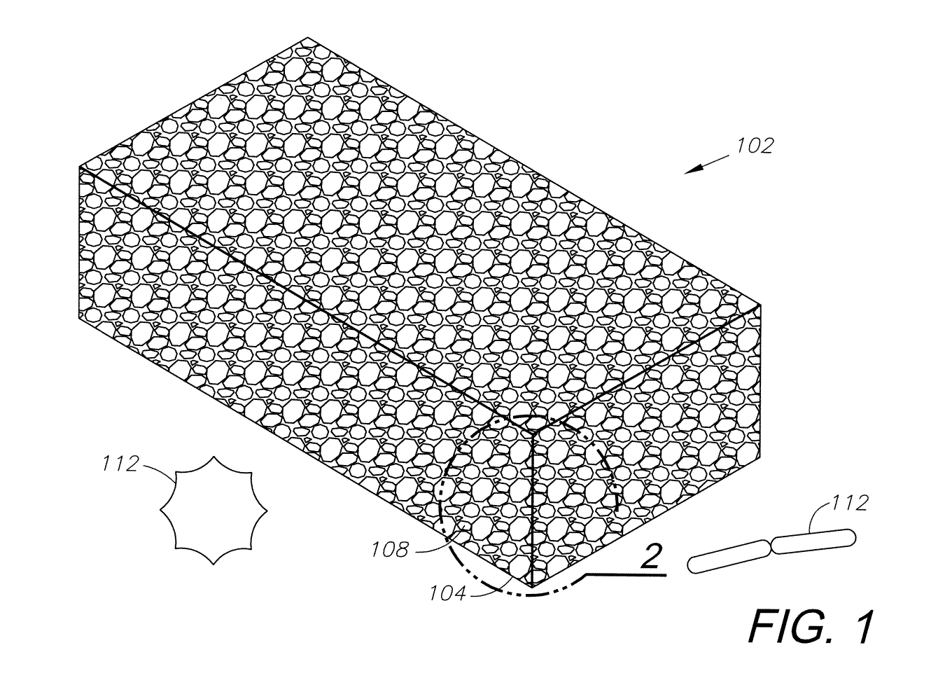

Referring to the drawings, FIG. 1 shows a resilient antimicrobial foam material 102 embodying principles of the disclosed subject matter having a plurality of cells 108. As described above, the resilient antimicrobial foam material 102 can be manufactured into a variety of resilient antimicrobial foam products. Therefore, the resilient antimicrobial foam material 102 shown in FIG. 1 is represented by a block of material for showing and describing the principles of the disclosed subject matter and is in no way to be interpreted as limiting. The resilient antimicrobial foam material 102 is shown adjacent to microbes 112 on a surface.

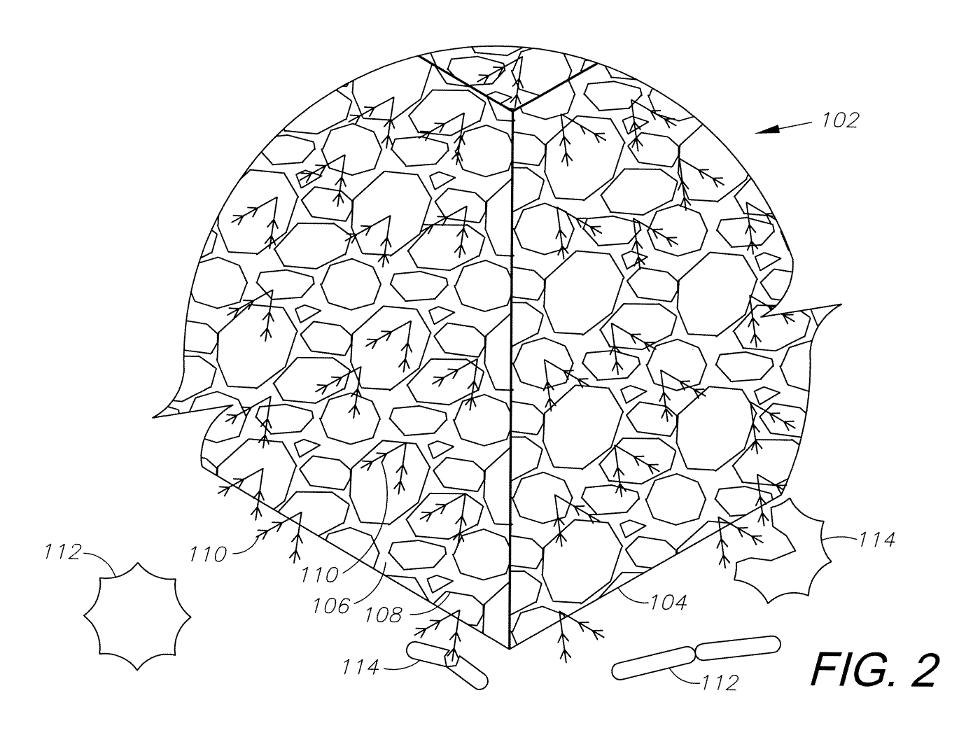

Referring to FIG. 2, active antimicrobial nanostructures 110 are shown attached to and projecting from the exterior surface 106 of a resilient foam material 104. The exterior surface 106 includes a surface of the resilient foam material 104 created by an exposed structural component of the resilient foam material 104 resulting from the shaping of the material, as well as by sectioning a cell 108 within the material. Because the active antimicrobial nanostructures 110 remain incorporated within the resilient foam material 104, the antimicrobial 110 is found incorporated within the structural component of the resilient foam material 104, projects from the exterior surface 106, as well as projects from the surfaces of the plurality of cells 108 disposed throughout the resilient foam material 104 that do not represent an exterior surface 106. Active and virulent microbes 112 present on a surface to be cleaned are brought into contact with the active antimicrobial nanostructures 110 causing the microbes to become incapacitated or destroyed microbes 114. Virulent microbes include gram positive and gram negative bacteria, fungi, algae, and yeast. Once the microbes are incapacitated or destroyed they are no longer virulent microbes. Because the nanostructure antimicrobials 110 remain incorporated within the resilient foam material 104 and incapacitate or destroy microbes by physical means, the incapacitated or destroyed microbes 114 either remain with the surface to be cleaned or become attached to the resilient antimicrobial foam material 102.

In use, the resilient antimicrobial foam material may be manufactured into a resilient antimicrobial foam product that may be wiped across a surface having microbes attached to it resulting in a demonstrable reduction of viable microbes on that surface. The antimicrobial nanostructure remains attached to the resilient antimicrobial foam material and is not left behind on the surface. Further, the antimicrobial does not leech out of the resilient foam material so there is no chemical residue left on the surface being cleaned.

The resilient antimicrobial foam material can be used to manufacture various resilient antimicrobial foam products, including sponges, pads, squeegees, cloths, gaskets, seals, coverings, handle grips, wound dressings, or any of a variety of foam products available. Further, membranes may be manufactured from the resilient antimicrobial foam material for use as a sterile barrier for a surface for example, in a medical or scientific environment. The resilient antimicrobial foam membrane becomes an antimicrobial barrier because a microbe coming into contact with the membrane would be incapacitated or destroyed by the antimicrobial nanostructure attached to the surface of the membrane because the nanostructure will pierce or lyse the microbe.

The resilient antimicrobial foam material has an additional benefit of being resistant to structural degradation and color loss due to the presence of the antimicrobial nanostructure and its ability to incapacitate or destroy microbes that are the causative agents of deterioration.

Resilient antimicrobial foam materials may be cleaned of any attached microbes and reused. After use, the resilient antimicrobial foam material may have microbes attached to its surface that were removed from the surface cleaned. The microbes may be removed using a wetting agent such as water or alcohol. Even without cleaning, most of the microbes remaining on the resilient antimicrobial foam material will be non-viable due to the antimicrobial nanostructure incorporated into the resilient foam material, and attached to any surface of the foam material.

### Example 1

Incorporation of nanostructure antimicrobials into resilient foam products according to the disclosed subject matter and use of the resilient antimicrobial foam material to mechanically clean virulent microbes from a surface has shown to create a marked decrease in virulent microbes on the surface because the method of incorporating the nanostructure antimicrobial into the foam polymer unexpectedly yields a resilient foam material whereby the nanostructure of the antimicrobial is maintained.

A resilient antimicrobial foam product was used to mechanically wipe multiple test areas in a child care facility to determine the effect of the product on removing, incapacitating, or destroying the microbes in the test area. The tests were conducted without adding a wetting agent to the surface or foam product. Tests were conducted in six areas, twice a day, for two days. The amount of viable pathogens were measured before and after cleaning the area with a resilient antimicrobial foam product by swabbing the test area, and analyzing the swab in a sampling device to measure the amount of contamination present. Microbes contain adenosine triphosphate, a biological energy molecule. The amount of contamination present on the swab was measured by assessing the adenosine triphosphate present using relative light units (RLU) generated in response to this activity. The RLUs were measured using a System Sure II™ luminometer (Hygiena, Camarillo, Calif.). The results shown in Table 1 represent the change in amount of contamination before and after cleaning a surface with the resilient antimicrobial foam material where a decrease is shown in parenthesis.

TABLE 1
Effect of mechanically cleaning surfaces in a child care
facility with a resilient antimicrobial foam material.

| | | | Microbe Count (RLU) | | |
|---|---|---|---|---|---|
| | | | Before | After | Percent |
| Area Tested | Day | Time | Cleaning | Cleaning | Change |
| Flooring at Entrance 1 | 1 | A.M. | 4573 | 679 | (85.152) |
| | 1 | P.M. | 1760 | 343 | (80.511) |
| | 2 | A.M. | 662 | 173 | (73.867) |
| | 2 | P.M. | 4622 | 668 | (85.547) |
| Flooring at Changing | 1 | A.M. | 1662 | 319 | (80.806 |
| Area | 1 | P.M. | 733 | 299 | (59.209) |
| | 2 | A.M. | 198 | 142 | (56.549) |
| Flooring at Child Play | 1 | A.M. | 2022 | 263 | (86.993) |
| Area | 1 | P.M. | 1089 | 153 | (85.950) |
| | 2 | A.M. | 481 | 209 | (56.549) |
| | 2 | P.M. | 2325 | 372 | (84.000) |
| Flooring at Crawl | 1 | A.M. | 804 | 41 | (94.900) |
| Area | 1 | P.M. | 2472 | 1774 | (28.236) |
| | 2 | A.M. | 407 | 231 | (43.243) |
| | 2 | P.M. | 2027 | 1116 | (44.943) |
| Food Prep & Sink | 1 | A.M. | 544 | 201 | (63.051) |
| Area | 1 | P.M. | 1402 | 1208 | (13.837) |
| | 2 | A.M. | 606 | 224 | (63.036) |
| Flooring at Entrance 2 | 1 | A.M. | 3 | 130 | 4233.333 |
| | 1 | P.M. | 2367 | 785 | (66.836) |
| | 2 | A.M. | 238 | 153 | (35.714) |
| | 2 | P.M. | 2048 | 1546 | (24.512) |

In the experiment above, mechanically cleaning a surface with an antimicrobial foam product embodying principles of the disclosed subject matter has shown to markedly decrease the presence of virulent microbes on the surface.

It will be appreciated that the resilient antimicrobial foam material can be used for various other applications. Moreover, the resilient antimicrobial foam material can be fabricated in various sizes and from a wide range of suitable materials, using various fabrication techniques.

1. A foam material, comprising:
a resilient foam material having a surface;
a plurality of cells formed within the resilient foam material;
an active antimicrobial bound to the resilient foam wherein an atomic nanostructure of the active antimicrobial projects from the surface.

2. The foam material of claim 1, wherein the resilient foam material is formed by extrusion.

3. The foam material of claim 1, wherein the resilient foam material is formed by vulcanization.

4. The foam material of claim 1, wherein the active antimicrobial mechanically incapacitates a microbe by lysing the microbe.

5. The foam material of claim 1, wherein the active antimicrobial mechanically incapacitates a microbe by piercing the microbe.

6. The foam material of claim 1, wherein the foam material is a foam product.

7. The foam material of claim 6, wherein the foam product is a membrane.

8. The foam material of claim 6, wherein the foam product is a sponge.

9. The foam material of claim 6, wherein the foam product is a wound dressing.

10. A method of manufacturing an antimicrobial foam, comprising the steps of:
providing a polymer material;
providing an active antimicrobial wherein an atomic nanostructure of the antimicrobial can mechanically incapacitate a microbe;
combining the polymer material with the antimicrobial;
heating the polymer material and the antimicrobial to form a hot melt;
pressurizing the hot melt;
incorporating a blowing agent into the hot melt;
cooling the hot melt to form a cool melt;
extruding the cool melt; and
forming a resilient antimicrobial foam material with a surface wherein the active antimicrobial is incorporated into the foam material.

11. The method of claim 10, further comprising the step of forming the antimicrobial foam material wherein the active antimicrobial is attached to and projecting from the surface.

12. The method of claim 10, further including the step of heating the hot melt to between about 300 to about 350 degrees Fahrenheit.

13. The method of using an antimicrobial foam, comprising the steps of:
providing a resilient foam material having a surface incorporating an active antimicrobial wherein the atomic nanostructure of the antimicrobial projects from a surface of the foam material and can mechanically incapacitate a microbe;
wiping the antimicrobial foam across a surface containing microbes; and
removing microbes from the surface with the antimicrobial foam.
